SEQUENCE LISTING (87 sequences)

SEQ ID NO: 1 - DNA, 20 bp, Artificial Sequence
  Probe Sequence
caaattaaagatcagtttcc

SEQ ID NO: 2 - DNA, 19 bp, Artificial Sequence
  Probe Sequence
gaaaaatcagatcagtttc

SEQ ID NO: 3 - DNA, 18 bp, Artificial Sequence
  Probe Sequence
aaaattcagggaaatgat

SEQ ID NO: 4 - DNA, 20 bp, Artificial Sequence
  Probe Sequence
gttttcaatggttttcccaa

SEQ ID NO: 5 - DNA, 19 bp, Artificial Sequence
  Probe Sequence
gttttcaatgatcagtttc

SEQ ID NO: 6 - DNA, 18 bp, Artificial Sequence
  Probe Sequence
cacagattgatctgggca

SEQ ID NO: 7 - DNA, 18 bp, Artificial Sequence
  Probe Sequence
caccgaaagattgacttt

SEQ ID NO: 8 - DNA, 14 bp, Artificial Sequence
  Probe Sequence
catgcaggaaacca

SEQ ID NO: 9 - DNA, 18 bp, Artificial Sequence
  Probe Sequence
ttccagaagaatccaaag

SEQ ID NO: 10 - DNA, 18 bp, Artificial Sequence
  Probe Sequence
ccttgaacagctgtaaag

SEQ ID NO: 11 - DNA, 20 bp, Artificial Sequence
  Probe Sequence
ctttaataggattgactttg

SEQ ID NO: 12 - DNA, 15 bp, Artificial Sequence
  Probe Sequence
tccagaagggccacc

SEQ ID NO: 13 - DNA, 21 bp, Artificial Sequence
  Probe Sequence
caatctacaagattgactttg

SEQ ID NO: 14 - DNA, 19 bp, Artificial Sequence
  Probe Sequence
acctaataggcatgtcaac

SEQ ID NO: 15 - DNA, 15 bp, Artificial Sequence
  Probe Sequence
aatgggagtggaagc

SEQ ID NO: 16 - DNA, 13 bp, Artificial Sequence
  Probe Sequence
gcctggataaacc

SEQ ID NO: 17 - DNA, 19 bp, Artificial Sequence
  Probe Sequence
gaatcttcatataaacctc

SEQ ID NO: 18 - DNA, 14 bp, Artificial Sequence
  Probe Sequence
gccaaaaacacttc

SEQ ID NO: 19 - DNA, 16 bp, Artificial Sequence
  Probe Sequence
gagcacagataaacct

SEQ ID NO: 20 - DNA, 17 bp, Artificial Sequence
  Probe Sequence
aggtttcagatcagttt

SEQ ID NO: 21 - DNA, 16 bp, Artificial Sequence
  Probe Sequence
cttaacaggcatgtca

SEQ ID NO: 22 - DNA, 17 bp, Artificial Sequence
  Probe Sequence
ttcaacagatcagtttc

SEQ ID NO: 23 - DNA, 18 bp, Artificial Sequence
  Probe Sequence
gaagagaaggttttccca

SEQ ID NO: 24 - DNA, 17 bp, Artificial Sequence
  Probe Sequence
gaagatcaggcatgtca

SEQ ID NO: 25 - DNA, 17 bp, Artificial Sequence
  Probe Sequence
agtaagtgatcagtttc

SEQ ID NO: 26 - DNA, 16 bp, Artificial Sequence
  Probe Sequence
tgctggtgatcagttt

SEQ ID NO: 27 - DNA, 15 bp, Artificial Sequence
  Probe Sequence
cgattcagatcagtt

SEQ ID NO: 28 - DNA, 15 bp, Artificial Sequence
  Probe Sequence
gaccctagatcagtt

SEQ ID NO: 29 - DNA, 17 bp, Artificial Sequence
  Probe Sequence
tgatagagatcagtttc

SEQ ID NO: 30 - DNA, 18 bp, Artificial Sequence
  Probe Sequence
cctatacagataaacctc

SEQ ID NO: 31 - DNA, 17 bp, Artificial Sequence
  Probe Sequence
gaaaacagataaacctc

SEQ ID NO: 32 - DNA, 14 bp, Artificial Sequence
  Probe Sequence
gaagcagataaacc

SEQ ID NO: 33 - DNA, 15 bp, Artificial Sequence
  Probe Sequence
ggttggagatcagtt

SEQ ID NO: 34 - DNA, 17 bp, Artificial Sequence
  Probe Sequence
gtattcagatcagtttc

SEQ ID NO: 35 - DNA, 17 bp, Artificial Sequence
  Probe Sequence
cacaggaagatcagttt

SEQ ID NO: 36 - DNA, 17 bp, Artificial Sequence
  Probe Sequence
cttaacagatcagtttc

SEQ ID NO: 37 - DNA, 18 bp, Artificial Sequence
  Probe Sequence
gattccaagatcagtttc

SEQ ID NO: 38 - DNA, 20 bp, Artificial Sequence
  Probe Sequence
ggaaactgatctttaatttg

SEQ ID NO: 39 - DNA, 19 bp, Artificial Sequence
  Probe Sequence
gaaactgatctgatttttc

SEQ ID NO: 40 - DNA, 18 bp, Artificial Sequence
  Probe Sequence
atcatttccctgaatttt

SEQ ID NO: 41 - DNA, 20 bp, Artificial Sequence
  Probe Sequence
ttgggaaaaccattgaaaac

SEQ ID NO: 42 - DNA, 19 bp, Artificial Sequence
  Probe Sequence
gaaactgatcattgaaaac

SEQ ID NO: 43 - DNA, 18 bp, Artificial Sequence
  Probe Sequence
tgcccagatcaatctgtg

SEQ ID NO: 44 - DNA, 18 bp, Artificial Sequence
  Probe Sequence
aaagtcaatctttcggtg

SEQ ID NO: 45 - DNA, 14 bp, Artificial Sequence
  Probe Sequence
tggtttcctgcatg

SEQ ID NO: 46 - DNA, 18 bp, Artificial Sequence
  Probe Sequence
ctttggattcttctggaa

SEQ ID NO: 47 - DNA, 18 bp, Artificial Sequence
  Probe Sequence
ctttacagctgttcaagg

SEQ ID NO: 48 - DNA, 20 bp, Artificial Sequence
  Probe Sequence
caaagtcaatcctattaaag

SEQ ID NO: 49 - DNA, 15 bp, Artificial Sequence
  Probe Sequence
ggtggcccttctgga

SEQ ID NO: 50 - DNA, 21 bp, Artificial Sequence
  Probe Sequence
caaagtcaatcttgtagattg

SEQ ID NO: 51 - DNA, 19 bp, Artificial Sequence
  Probe Sequence
gttgacatgcctattaggt

SEQ ID NO: 52 - DNA, 15 bp, Artificial Sequence
  Probe Sequence
gcttccactcccatt

SEQ ID NO: 53 - DNA, 13 bp, Artificial Sequence
  Probe Sequence
ggtttatccaggc

SEQ ID NO: 54 - DNA, 19 bp, Artificial Sequence
  Probe Sequence
gaggtttatatgaagattc

SEQ ID NO: 55 - DNA, 14 bp, Artificial Sequence
  Probe Sequence
gaagtgtttttggc

SEQ ID NO: 56 - DNA, 16 bp, Artificial Sequence
  Probe Sequence
aggtttatctgtgctc

SEQ ID NO: 57 - DNA, 17 bp, Artificial Sequence
  Probe Sequence
aaactgatctgaaacct

SEQ ID NO: 58 - DNA, 16 bp, Artificial Sequence
  Probe Sequence
tgacatgcctgttaag

SEQ ID NO: 59 - DNA, 17 bp, Artificial Sequence
  Probe Sequence
gaaactgatctgttgaa

SEQ ID NO: 60 - DNA, 18 bp, Artificial Sequence
  Probe Sequence
tgggaaaaccttctcttc

SEQ ID NO: 61 - DNA, 17 bp, Artificial Sequence
  Probe Sequence
tgacatgcctgatcttc

SEQ ID NO: 62 - DNA, 17 bp, Artificial Sequence
  Probe Sequence
gaaactgatcacttact

SEQ ID NO: 63 - DNA, 16 bp, Artificial Sequence
  Probe Sequence
aaactgatcaccagca

SEQ ID NO: 64 - DNA, 15 bp, Artificial Sequence
  Probe Sequence
aactgatctgaatcg

SEQ ID NO: 65 - DNA, 15 bp, Artificial Sequence
  Probe Sequence
aactgatctagggtc

SEQ ID NO: 66 - DNA, 17 bp, Artificial Sequence
  Probe Sequence
gaaactgatctctatca

SEQ ID NO: 67 - DNA, 18 bp, Artificial Sequence
  Probe Sequence
gaggtttatctgtatagg

SEQ ID NO: 68 - DNA, 17 bp, Artificial Sequence
  Probe Sequence
gaggtttatctgttttc

SEQ ID NO: 69 - DNA, 14 bp, Artificial Sequence
  Probe Sequence
ggtttatctgcttc

SEQ ID NO: 70 - DNA, 15 bp, Artificial Sequence
  Probe Sequence
aactgatctccaacc

SEQ ID NO: 71 - DNA, 17 bp, Artificial Sequence
  Probe Sequence
gaaactgatctgaatac

SEQ ID NO: 72 - DNA, 17 bp, Artificial Sequence
  Probe Sequence
aaactgatcttcctgtg

SEQ ID NO: 73 - DNA, 17 bp, Artificial Sequence
  Probe Sequence
gaaactgatctgttaag

SEQ ID NO: 74 - DNA, 18 bp, Artificial Sequence
  Probe Sequence
gaaactgatcttggaatc

SEQ ID NO: 75 - DNA, 40 bp, Homo sapiens
aaagagaaagcaaattaaagatcagtttcctaattcatct

SEQ ID NO: 76 - DNA, 26 bp, Artificial Sequence
  Primer Sequence
aagagaaagcaaattaaagatcagtt

SEQ ID NO: 77 - DNA, 19 bp, Artificial Sequence
  Primer Sequence
ctgtcagaggatactgcac

SEQ ID NO: 78 - DNA, 19 bp, Artificial Sequence
  Primer Sequence
gttttcccagtcacgacgt

SEQ ID NO: 79 - DNA, 21 bp, Artificial Sequence
  Primer Sequence
gcaaatggcattctgacatcc

SEQ ID NO: 80 - DNA, 22 bp, Artificial Sequence
  Primer Sequence
gcggataacaatttcacacagg

SEQ ID NO: 81 - DNA, 17 bp, Artificial Sequence
  Primer Sequence
cgtccatgccgagagtg

SEQ ID NO: 82 - DNA, 23 bp, Artificial Sequence
  Primer Sequence
ctttatgtttttggcgtcttcca

SEQ ID NO: 83 - DNA, 21 bp, Artificial Sequence
  Primer Sequence
gactggttccaattgacaagc

SEQ ID NO: 84 - DNA, 19 bp, Artificial Sequence
  Primer Sequence
gcgtgaatgtaagcgtgac

SEQ ID NO: 85 - DNA, 18 bp, Artificial Sequence
  Primer Sequence
tgtaaaacgacggccagt

SEQ ID NO: 86 - DNA, 20 bp, Artificial Sequence
  Primer Sequence
aagggtcttgcgaaggatag

SEQ ID NO: 87 - DNA, 23 bp, Artificial Sequence
  Primer Sequence
gggttatgctagttattgctcag

(19) United States
(12) Patent Application Publication
(10) Pub. No.: US 2022/0259671 A1
(43) Pub. Date: Aug. 18, 2022

(54) KIT AND METHODS TO DETECT MET GENE FUSION

(71) Applicants: AN HSU, Taipei (TW); PEI-YI LIN, Taipei (TW); WAN-TING HUANG, Taipei (TW); DATSEN GEORGE WEI, Taipei (TW); SHU-JEN CHEN, Taipei (TW); HUA-CHIEN CHEN, Taipei (TW)

(72) Inventors: AN HSU, Taipei (TW); PEI-YI LIN, Taipei (TW); WAN-TING HUANG, Taipei (TW); DATSEN GEORGE WEI, Taipei (TW); SHU-JEN CHEN, Taipei (TW); HUA-CHIEN CHEN, Taipei (TW)

(21) Appl. No.: 17/624,845

(22) PCT Filed: Aug. 28, 2020
(86) PCT No.: PCT/US20/48332

§ 371 Date: Jan. 5, 2022

Related U.S. Application Data
(60) Provisional application No. 62/893,151, filed on Aug. 28, 2019.

(51) Int. Cl. C12Q 1/6886

(52) U.S. Cl. CPC C12Q 1/6886; C12Q 1/6818

(57) ABSTRACT

Provided is a kit for detecting MET fusion, including MET gene fusion and MET exon 14 skipping. The kit includes a set of MET fusion-specific primer pairs and a set of MET fusion-specific probes. Also provided is a method for detecting MET fusion, including generating an amplified target cDNA to hybridize with the set of MET fusion-specific probes in a single reaction and detecting the probe-bound product to identify all possible MET fusions in a biological sample.

### CROSS-REFERENCE TO RELATED APPLICATIONS

This application claims priority of Provisional Application No. 62/893,151, filed on Aug. 28, 2019, the content of which is incorporated herein in its entirety by reference.

### FIELD OF THE INVENTION

The invention relates to a kit and a method for molecular diagnostics and genomics. Particularly, the invention relates to a kit and a method for molecular diagnostics and genomics of cancers.

### SEQUENCE LISTING

The instant application contains a Sequence Listing which has been submitted electronically in ASCII format and is hereby incorporated by reference in its entirety. Said ASCII copy, created Aug. 28, 2020, is named “ACTG-4PCT_SEQList_ST25.txt” and is 16,606 bytes in size.

### BACKGROUND OF THE INVENTION

MET proto-oncogene, receptor tyrosine kinase (MET) is a gene codes for a receptor tyrosine kinase with its ligand being hepatocyte growth factor. Upon binding to its ligand, the MET receptor is activated and triggers signals that promote cell survival, embryogenesis, cell migration, and invasion.

Genomic alterations in MET may lead to overexpression of the MET receptor and/or its activation independent of its ligand. One of such alterations is MET gene fusion, which is a fusion between at least a part of MET gene and a part of a highly expressed partner gene. Importantly, more than a hundred different mutations can result in exon 14 skipping in the MET gene. MET exon 14 skipping is caused by aberrant splicing, which leads to fusion between exon 13 and exon 15 of the MET gene and ultimately leads to deletion of the transmembrane domain of the MET receptor. These alterations enhance the signaling of the MET receptor pathway, ultimately leading to cancers.

Cancer patients harboring MET fusion such as MET gene fusion or MET exon 14 skipping show treatment response to MET inhibitor therapies. However, MET gene fusion occurs at low frequencies and the prevalence of MET exon 14 skipping is only 3-4% in lung cancer. Therefore, it is important to screen cancer patients for MET fusion and identify the ones who may benefit from MET inhibitors before treatment.

Several technologies may be adopted for MET fusion detection, including next-generation sequencing (NGS), Sanger sequencing, and reverse transcription polymerase chain reaction (RT-PCR). While NGS provides a large amount of data, the data analysis process itself represents a huge burden to clinical use, which requires high efficiency and simplicity. Sanger sequencing can be used to detect single gene alterations, but its sensitivity can be hampered by large deletions or low allele frequency of the gene alterations. RT-PCR has high sensitivity, but it requires different probes and separate reactions for detecting each fusion type, resulting in the need for more samples as the number of fusion types to be detected increases. Because dozens of MET fusion types have been discovered, there is a demand for novel methods to detect MET fusion, including MET gene fusion and MET exon 14 skipping in one reaction.

### SUMMARY OF THE INVENTION

The present disclosure concerns a method for detecting MET fusion. The method includes the steps of:
(a) obtaining ribonucleic acids (RNA) from a biological sample;
(b) performing reverse transcription of the RNA to obtain complementary deoxyribonucleic acids (cDNA);
(c) amplifying the cDNA with at least two MET fusion-specific primer pairs to obtain an amplified product;
(d) mixing the amplified product with at least two probes to obtain a probe-bound product, wherein each of the probes has a different nucleotide sequence selected from the group consisting of SEQ ID NOs:1-37 and any complementary sequence thereof; and
(e) detecting the probe-bound product to determine the presence of MET fusion.

The disclosed method utilizes a set of specifically designed probes, each of which can capture the amplified product including one particular MET fusion sequence, to detect all possible MET fusions. Since the MET fusion types are numerous, but the exact one or more MET fusions in the biological sample are unknown before the detection step, it is important to obtain detectable amounts of the amplified products for all types of MET fusions so that any MET fusion type can be detected subsequently. Considering that different MET fusion-specific primer pairs show different amplification efficiencies, an additional round of DNA amplification may be performed in the aforementioned step (c) using a pair of universal primers so as to ensure sufficient production of the amplified product of any MET fusion type. Accordingly, in some preferred embodiments, in step (c) the cDNA is amplified first with the at least two MET fusion-specific primer pairs and subsequently with a universal primer pair to obtain the amplified product. In this setting, a MET fusion-specific forward primer in each of the MET fusion-specific primer pairs further encompasses the nucleotide sequence of a universal forward primer in the universal primer pair, and a MET fusion-specific reverse primer in each of the MET fusion-specific primer pairs further encompasses the nucleotide sequence of a universal reverse primer in the universal primer pair.

In another aspect, a kit is also provided for detecting MET fusion according to the aforementioned method. The kit includes at least two MET fusion-specific primer pairs; and at least two probes each having a different nucleotide sequence selected from the group consisting of SEQ ID NOs:1-37 and any complementary sequence thereof.

In some preferred embodiments, the kit further includes a universal primer pair for an additional round of DNA amplification. In this setting, a MET fusion-specific forward primer in each of the MET fusion-specific primer pairs further encompasses the nucleotide sequence of a universal forward primer in the universal primer pair, and a MET fusion-specific reverse primer in each of the MET fusion-specific primer pairs further encompasses the nucleotide sequence of a universal reverse primer in the universal primer pair.

In some embodiments, the kit further includes a reverse transcriptase for reverse transcription of the RNA isolated from the biological sample, and also includes a DNA polymerase for amplification of the cDNA generated by the reverse transcription.

The set of probes used in the method and the kit can target distinct MET fusions with high specificities and also show similar hybridization efficiencies, ensuring accurate detection of all possible MET fusions in one single reaction. Thus, application of the method and the kit in MET fusion detection can reduce the need for large sample volume and saves detection time due to no requirement for separate detections of different MET fusion types.

In addition, the method and the kit are compatible with the platforms designed for multiplex reactions.

### BRIEF DESCRIPTION OF THE DRAWINGS

The disclosure will be apparent to those skilled in the art from the following detailed description of the preferred embodiments, with reference to the attached drawings, in which:

FIG. 1 is a schematic illustration of the method for detecting MET fusion according to one embodiment of the invention; the detection is based on a single-amplified target-probe-barcoded magnetic bead (BMB) hybridization assay; and

FIG. 2 is a schematic illustration of the method for detecting MET fusion according to one embodiment of the invention; the detection is based on a double-amplified target-probe hybridization assay.

### DETAILED DESCRIPTION

Unless defined otherwise, all technical and scientific terms used herein have the same meaning as commonly understood by a person skilled in the art to which this disclosure belongs.

### Definition

As used herein, the singular forms “a”, “an”, and “the” include plural referents unless the context clearly dictates otherwise.

As used herein, the term “MET fusion” refers to the MET alterations including MET gene fusions and MET exon 14 skipping. “MET exon 14 skipping” refers to the phenomenon where the 3′ or 5′ splice sites of exon 14 in the MET gene mutate to cause exclusion of exon 14 from the mRNA, leading to a fusion between exon 13 and exon 15 in the MET RNA transcript.

The term “gene fusion” refers to a phenomenon where a first gene on a chromosome is fused to a second gene on the same or a different chromosome and a hybrid gene or a fusion gene thus forms. This phenomenon is also commonly referred to as “gene translocation” or “gene rearrangement.” When the MET gene is one of the multiple fused genes, such gene fusion is called “MET gene fusion”. Typically, two “partner genes” are fused together. The gene at the 5′ end of the fusion gene is referred to as “5′ partner” or “5′ gene”, and the gene at the 3′ end of the fusion gene is referred to as “3′ partner” or “3′ gene.” The fusion gene has a “fusion junction,” which is the site where the 5′ partner gene fuses to the 3′ partner gene. Fusion junction is located in a fusion region defined by a fusion sequence (also called a fusion junction sequence), which encompasses the sequence from the 5′ gene and the sequence from the 3′ gene. Different combinations of partner genes lead to different “fusion types.” The fusion between two specific genes is further diversified by fusion junction since fusion junction may occur anywhere within the partner genes. For example, the fusion between the first exon of a first gene and the second exon of a second gene is one fusion type, whereas the fusion between the third exon of the first gene and the first exon of the second gene is another fusion type. In this disclosure, MET exon 14 skipping is considered one fusion type, and the MET gene having such mutation is considered one MET fusion gene, with the 5′ partner encompassing exon 1 to exon 13 of the MET gene and the 3′ partner encompassing exon 15 to exon 20 of the MET gene.

Gene fusions may be detected by identifying a fusion junction in a DNA or in an RNA transcript of that DNA. As used herein, a “fusion type” refers to a unique fusion present in an RNA transcript. In other words, it is considered the same fusion type when the fusions between two specific genes occur at different sites within the same intronic region. For example, a fusion between exon 3 of gene A and exon 5 of gene B may have a DNA fusion region containing a small portion of the intron between exons 3 and 4 of gene A and a large portion of the intron between exons 4 and 5 of gene B. Alternatively, such fusion may have a DNA fusion region containing a large portion of the intron between exons 3 and 4 of gene A and a small portion of the intron between exons 4 and 5 of gene B. These two fusions, though having different DNA fusion junctions, are considered the same “fusion type” because the RNA transcripts generated from the two fusions are the same.

The term “primer” refers to a synthetic single-stranded oligonucleotide that can be used to amplify a target nucleic acid having a specific length. As used herein, the terms “MET fusion-specific primer” and “fusion specific primer” are used interchangeably, which refer to a DNA primer that is designed to amplify a target cDNA including a fusion junction originates from a particular MET fusion gene. The MET fusion-specific primers are used in pairs, including a MET fusion-specific forward primer capable of specifically binding to the 5′-end of a target cDNA, and a MET fusion-specific reverse primer capable of specifically binding to the 3′-end of said target cDNA.

As used herein, the term “universal primer” refers to a DNA primer that is designed to amplify any DNA including the nucleotide sequence of the universal primer. The universal primers are used in pairs, including a universal forward primer and a universal reverse primer.

Unless defined otherwise, the term “probe” or “MET fusion-specific probe” refers to a synthetic single-stranded DNA oligonucleotide that can hybridize to a fusion region originates from a particular MET fusion gene.

As used herein, a “connector” or a “linker” refers to part of a molecule or part of a complex of molecules that connects one molecule to another. The connector or linker can act by covalent bonding, nucleic acid hybridization, or non-covalent interaction between a pair of molecules such as biotin-streptavidin interaction. In this disclosure, a “connector” is used to conjugate a primer with a detectable molecule such as a fluorescent molecule; and a “linker” is used to form linkage between a probe and a detectable molecule or a unique identifier such as a barcoded magnetic bead (BMB).

In the present disclosure, a method for detecting MET fusion is provided. The method includes the steps of:
(a) obtaining RNA from a biological sample;
(b) performing reverse transcription of the RNA to obtain cDNA;
(c) amplifying the cDNA with at least two MET fusion-specific primer pairs to obtain an amplified product;
(d) mixing the amplified product with at least two probes to obtain a probe-bound product, wherein each of the probes has a different nucleotide sequence selected from the group consisting of SEQ ID NOs: 1-37 and any complementary sequence thereof; and
(e) detecting the probe-bound product to determine the presence of MET fusion.

In step (a) of the disclosed method, RNA is prepared from a biological sample. The biological sample may be any sample obtained from an animal and a human subject. Examples of the biological samples include a formalin-fixed paraffin-embedded (FFPE) tissue section, blood, plasma, or cells. In some embodiments, the biological sample originates from a cancer patient. In some embodiments, the biological sample originates from a solid tumor, soft tissue sarcoma, or a hematological cancer. In some embodiments, the biological sample originates from a patient with lung cancer, breast cancer, colorectal cancer, endometrial cancer, gastric cancer, malignant solid tumor, head and neck cancer, glioblastoma, hepatocellular carcinoma, lymphoma, or multiple myeloma.

Preparation of total RNA from the biological sample can be carried out by various methods known in the art. One typical procedure is RNA extraction with organic solvents such as phenol/chloroform and precipitation by centrifugation. There are also commercially available kits for RNA isolation or purification. Once the RNA is obtained, a reverse transcriptase is used along with four kinds of deoxyribonucleoside triphosphates (dNTP, including dATP, dCTP, dTTP, and dGTP) to generate cDNA from the template RNA, a process called reverse transcription. The reverse transcription may be conducted using SuperScript cDNA synthesis kit (Cat No: 11754050, Invitrogen).

In step (c) of the disclosed method, the cDNA is amplified with a DNA polymerase and at least two pairs of MET fusion-specific primers to obtain an amplified product for probe detection. The amplification may be conducted using a multiplex PCR kit (Cat No: 206143, Qiagen) which includes a DNA polymerase. The MET fusion-specific primers may be provided as a regent before use. In some embodiments, all the MET fusion-specific primer pairs are pooled together to form a single pooled reagent. In other embodiments, the MET fusion-specific primer pairs are partially pooled to form a plurality of pooled reagents, each of which contains at least one MET fusion-specific primer pairs. Thus, the number of the pooled reagent may be 1, 2, 3, 4, 5, 6, 7, 8, 9, 10 or more. In some preferred embodiments, the dozens of MET fusion-specific primer pairs are provided in two pooled reagents to be used in two multiplex amplification reactions. DNA amplification in this manner has been demonstrated to show significantly higher performance than a single multiplex amplification reaction carried out with all the MET fusion-specific primer pairs, probably due to decreased primer complexity.

In some preferred embodiments, in step (c) the cDNA is amplified first with the at least two MET fusion-specific primer pairs and subsequently with a universal primer pair to obtain the amplified product. When the universal primer pair is utilized in the disclosed method, the MET fusion-specific forward primer in each of the MET fusion-specific primer pairs further encompasses the nucleotide sequence of the universal forward primer in the pair of universal primers, and the MET fusion-specific reverse primer in each of the MET fusion-specific primer pairs further encompasses the nucleotide sequence of the universal reverse primer in the universal primer pair. The use of the universal primer pair can increase the ultimate yield of any possible amplified product, no matter which MET fusion type is to be detected or which MET fusion-specific primer pair is used in the first round of amplification. An additional advantage of applying the universal primer pair is that when primers are to be modified to become detectable, only two or even one universal primer needs to be modified, for example, forming a linkage between one universal primer and a connector (such as biotin) so that a detectable molecule can be linked to the universal primer. Otherwise, all MET fusion-specific primer pairs have to be modified, which makes the primer modification process more complicated and costly.

In step (d) of the disclosed method, the amplified product is mixed with at least two probes so that a probe-bound product can form through nucleic acid hybridization. Because the probes are specifically designed based on the fusion sequence of each type of MET fusion, the exact MET fusion type can be determined by detecting the particular probe-bound product. The MET fusion that can be detected includes, but not limited to, a fusion between exons 13 and 15 of the MET gene, or a fusion between the MET gene and a partner gene selected from C8orf34 (encoding chromosome 8 open reading frame 34), CAPZA2 (encoding capping actin protein of muscle Z-line subunit alpha 2), KIF5B (encoding kinesin family member 5B), CAV1 (encoding caveolin 1), DYNC1I1 (encoding dynein cytoplasmic 1 intermediate chain 1), WNT2 (encoding Wnt family member 2), TFG (encoding trafficking from ER to golgi regulator), MIR548F1 (encoding microRNA 548f-1), OXR1 (encoding oxidation resistance 1), PTPRZ1 (encoding protein tyrosine phosphatase receptor type Z1), ST7 (encoding suppression of tumorigenicity 7), TPR (encoding translocated promoter region, nuclear basket protein), BAIAP2L1 (encoding BAR/IMD domain containing adaptor protein 2 like 1), CLIP2 (encoding CAP-Gly domain containing linker protein 2), DCTN1 (encoding dynactin subunit 1), EPS15 (encoding epidermal growth factor receptor pathway substrate 15), LRRFIP1 (encoding LRR binding FLII interacting protein 1), PPFIBP1 (encoding PPFIA binding protein 1), SLC34A2 (encoding solute carrier family 34 member 2), and TRIM4 (encoding tripartite motif containing 4). In one preferred embodiment, the detectable MET fusions include CAPZA2-MET fusions.

Table 1 lists the specifically designed probes for detection of the indicated MET fusions.

Each of these probes has been demonstrated to be specific to the indicated gene fusion type and does not cross-reacts with other fusion types, allowing accurate determination of the particular MET fusion type. In some embodiments, for detection of one particular MET fusion, both the probe having any of the sequence of SEQ ID NOs:1-74 and the probe having a complementary sequence are used to enhance detection efficiency. For example, for detection of MET fusion 001 in Table 1, two probes with the sequences of SEQ ID NO:1 and SEQ ID NO:38 may be used together.

TABLE 1

| MET | 5′ gene | 3′ gene | Probe Sequence | SEQ |
|---|---|---|---|---|
| fusion ID | (exon no.) | (exon no.) | (from the 5′-end to the 3′-end) | ID NO |
| 001 | MET | MET | CAAATTAAAGATCAGTTTCC | 1 |
| | (13) | (15) | GGAAACTGATCTTTAATTTG | 38 |
| 002 | C8orf34 | MET | GAAAAATCAGATCAGTTTC | 2 |
| | (2) | (15) | GAAACTGATCTGATTTTTC | 39 |
| 003 | CAPZA2 | MET | AAAATTCAGGGAAATGAT | 3 |
| | (7) | (12) | ATCATTTCCCTGAATTTT | 40 |
| 004 | CAPZA2 | MET | GTTTTCAATGGTTTTCCCAA | 4 |
| | (2) | (6) | TTGGGAAAACCATTGAAAAC | 41 |
| 005 | CAPZA2 | MET | GTTTTCAATGATCAGTTTC | 5 |
| | (2) | (15) | GAAACTGATCATTGAAAAC | 42 |
| 006 | KIF5B | MET | CACAGATTGATCTGGGCA | 6 |
| | (24) | (14) | TGCCCAGATCAATCTGTG | 43 |
| 007 | MET | CAV1 | CACCGAAAGATTGACTTT | 7 |
| | (1) | (3) | AAAGTCAATCTTTCGGTG | 44 |
| 008 | MET | DYNCH1 | CATGCAGGAAACCA | 8 |
| | (3) | (2) | TGGTTTCCTGCATG | 45 |
| 009 | MET | C8orf34 | TTCCAGAAGAATCCAAAG | 9 |
| | (14) | (3) | CTTTGGATTCTTCTGGAA | 46 |
| 010 | MET | WNT2 | CCTTGAACAGCTGTAAAG | 10 |
| | (16) | (4) | CTTTACAGCTGTTCAAGG | 47 |
| 011 | MET | CAV1 | CTTTAATAGGATTGACTTTG | 11 |
| | (2) | (3) | CAAAGTCAATCCTATTAAAG | 48 |
| 012 | MET | TFG | TCCAGAAGGGCCACC | 12 |
| | (14) | (6) | GGTGGCCCTTCTGGA | 49 |
| 013 | MET | CAV1 | CAATCTACAAGATTGACTTTG | 13 |
| | (5) | (3) | CAAAGTCAATCTTGTAGATTG | 50 |
| 014 | MIR548F1 | MET | ACCTAATAGGCATGTCAAC | 14 |
| | (1) | (11) | GTTGACATGCCTATTAGGT | 51 |
| 015 | OXR1 | MET | AATGGGAGTGGAAGC | 15 |
| | (11) | (13) | GCTTCCACTCCCATT | 52 |
| 016 | PTPRZ1 | MET | GCCTGGATAAACC | 16 |
| | (1) | (2) | GGTTTATCCAGGC | 53 |
| 017 | ST7 | MET | GAATCTTCATATAAACCTC | 17 |
| | (1) | (2) | GAGGTTTATATGAAGATTC | 54 |
| 018 | ST7 | MET | GCCAAAAACACTTC | 18 |
| | (11) | (3) | GAAGTGTTTTTGGC | 55 |
| 019 | ST7 | MET | GAGCACAGATAAACCT | 19 |
| | (1) | (2) | AGGTTTATCTGTGCTC | 56 |
| 020 | TFG | MET | AGGTTTCAGATCAGTTT | 20 |
| | (5) | (15) | AAACTGATCTGAAACCT | 57 |
| 021 | TPR | MET | CTTAACAGGCATGTCA | 21 |
| | (4) | (11) | TGACATGCCTGTTAAG | 58 |
| 022 | BAIAP2L1 | MET | TTCAACAGATCAGTTTC | 22 |
| | (9) | (15) | GAAACTGATCTGTTGAA | 59 |
| 023 | CAPZA2 | MET | GAAGAGAAGGTTTTCCCA | 23 |
| | (1) | (6) | TGGGAAAACCTTCTCTTC | 60 |
| 024 | CAPZA2 | MET | GAAGATCAGGCATGTCA | 24 |
| | (4) | (11) | TGACATGCCTGATCTTC | 61 |
| 025 | CLIP2 | MET | AGTAAGTGATCAGTTTC | 25 |
| | (11) | (15) | GAAACTGATCACTTACT | 62 |
| 026 | DCTN1 | MET | TGCTGGTGATCAGTTT | 26 |
| | (26) | (15) | AAACTGATCACCAGCA | 63 |
| 027 | EPS15 | MET | CGATTCAGATCAGTT | 27 |
| | (21) | (15) | AACTGATCTGAATCG | 64 |
| 028 | LRRFIP1 | MET | GACCCTAGATCAGTT | 28 |
| | (19) | (15) | AACTGATCTAGGGTC | 65 |
| 029 | PPFIBP1 | MET | TGATAGAGATCAGTTTC | 29 |
| | (9) | (15) | GAAACTGATCTCTATCA | 66 |
| 030 | PTPRZ1 | MET | CCTATACAGATAAACCTC | 30 |
| | (2) | (2) | GAGGTTTATCTGTATAGG | 67 |
| 031 | PTPRZ1 | MET | GAAAACAGATAAACCTC | 31 |
| | (3) | (2) | GAGGTTTATCTGTTTTC | 68 |
| 032 | PTPRZ1 | MET | GAAGCAGATAAACC | 32 |
| | (8) | (2) | GGTTTATCTGCTTC | 69 |
| 033 | SLC34A2 | MET | GGTTGGAGATCAGTT | 33 |
| | (4) | (15) | AACTGATCTCCAACC | 70 |
| 034 | TFG | MET | GTATTCAGATCAGTTTC | 34 |
| | (7) | (15) | GAAACTGATCTGAATAC | 71 |
| 035 | TPR | MET | CACAGGAAGATCAGTTT | 35 |
| | (36) | (15) | AAACTGATCTTCCTGTG | 72 |
| 036 | TPR | MET | CTTAACAGATCAGTTTC | 36 |
| | (4) | (15) | GAAACTGATCTGTTAAG | 73 |
| 037 | TRIM4 | MET | GATTCCAAGATCAGTTTC | 37 |
| | (6) | (15) | GAAACTGATCTTGGAATC | 74 |

In some embodiments, one MET fusion type is detected after one target cDNA is amplified and probed. In other embodiments, multiple MET fusion types can be detected simultaneously after two or more target cDNAs with difference sequences are amplified in one reaction (called a multiplex amplification reaction) and/or probed in one reaction (called a multiplex hybridization reaction). When the disclosed method is performed in the multiplex setting, at least two probes for detecting at least two MET fusion types are applied. In some embodiments, the at least two probes are selected from the group consisting of SEQ ID NOs:1-37 and any complementary sequence thereof. In some preferred embodiments, the at least two probes are selected from the group consisting of SEQ ID NOs:3, 4, 5, 23, 24 and any complementary sequence thereof. The probes may be provided as a single pooled reagent or as separate reagents.

Typically, the probe and the amplified product are mixed at a specific temperature to facilitate probe hybridization. The optimal thermo mixing condition for probe hybridization varies depending on probe sequences. Thus, for a multiplex reaction where at least two probes are applied, it is difficult to select a suitable hybridization condition for all the probes. However, by using the probes listed in Table 1, a multiplex reaction may be performed at a fixed temperature with agitation at a fixed speed, because these probes are designed to be capable of hybridizing to the respective target at similar hybridization conditions. In some embodiments, the temperature for hybridization is between 35-50° C., 40-50° C., 40-45° C., or 45-50° C. In some embodiments, the hybridization is performed by using a thermomixer at a rotation speed between 700-1000 rpm, 750-1000 rpm, 800-1000 rpm, 900-1000 rpm, 700-750 rpm, 700-800 rpm, 750-800 rpm, or 800-900 rpm.

Detection of the probe-bound product may be accomplished by detecting the MET fusion-specific primers, the universal primers, or the probe in said product. Thus, the primers or probes are usually modified to be detectable. They may be modified to have fluorescence or chemiluminescence activity or become chromogenic or colorimetric by being connected directly or indirectly to a detectable molecule. In some embodiments, one or both primers in the primer pair are connected to biotin or other compounds capable of binding to a streptavidin-conjugated detectable molecule. The detectable molecule may be a dye, a fluorescent molecule such as phycoerythrin (PE) or cyanines, or an enzyme for a chromogenic reaction such as alkaline phosphatase (AP) or horseradish peroxidase (HRP). The enzyme used in a chromogenic reaction catalyzes the production of colored compounds in the presence of a chromogenic substrate.

In some embodiments, the probe for detecting one particular MET fusion type is connected to a unique identifier such that multiple MET fusion types can be detected simultaneously and distinguished from one another. The unique identifier may be an oligonucleotide with a unique sequence, or a microbead or a nanoparticle that includes a unique barcode on the surface. The barcode may be a geometric pattern that can be read by an optical scanner with a brightfield imaging system. In some embodiments, the microbead or nanoparticle is a magnetic particle. In some embodiments, the microbead or nanoparticle is made of synthetic polymers.

The unique identifier may be connected to the probe directly or through a linker. In some embodiments, the unique identifier is connected to the probe by direct chemical coupling and a covalent bond is formed therebetween. In some embodiments, the unique identifier is connected to the probe through a polymer linker. In some embodiments, the unique identifier is connected to the probe by hybridization between complementary nucleotide sequences.

The disclosed method can be performed on several technology platforms capable of running multiplex reactions, such as a microarray plate, a gene chip, microbeads, nanoparticles, a membrane, or a microfluidic device. In some embodiments, the probes are immobilized on a microarray plate, a gene chip, or a membrane at different positions, for example, in the form of an array of spots, each containing multiple copies of one type of probe. In other embodiments, the probes are coupled with microbeads (such as micro magnetic beads). In still other embodiments, the probes are coated on a substrate plate of a microfluidic device, in which different probes are placed in different regions of the substrate plate.

When the probes are immobilized on a DNA microarray plate, the microarray plate may further include a set of control spots, each containing multiple copies of a control probe. The control probe binds the cDNA of housekeeping genes such as beta-actin, glyceraldehyde 3-phosphate dehydrogenase (GAPDH), and beta 2-microglobulin. Thus, the control spots can be used as an internal control to validate assay performance. In addition, the microarray plate may further include a set of anchor spots, each containing multiple copies of an anchor probe. The anchor probe is designed to be detected irrespective of the amplified products. Thus, the anchor spots can be used as a position indicator for nearby spots on the microarray plate.

In the present disclosure, a kit is also provided for detecting MET fusion according to the disclosed method. The kit includes at least two MET fusion-specific primer pairs; and at least two probes each having a different nucleotide sequence selected from the group consisting of SEQ ID NOs:1-37 and any complementary sequence thereof.

In some preferred embodiments, the kit further includes a universal primer pair. When the

MET fusion-specific primer pairs are used in combination with the universal primer pair, the MET fusion-specific forward primer in each of the MET fusion-specific primer pairs further encompasses the nucleotide sequence of the universal forward primer in the universal primer pair, and the MET fusion-specific reverse primer in each of the MET fusion-specific primer pairs further encompasses the nucleotide sequence of the universal reverse primer in the universal primer pair.

In some embodiments, the kit further includes a reverse transcriptase for reverse transcription of the RNA isolated from the biological sample, and also includes a DNA polymerase for amplification of the cDNA generated by the reverse transcription.

In some embodiments, the kit further includes an internal control. The internal control may be a positive control sample where a MET gene fusion or MET exon 14 skipping is present or may be a negative control sample having no MET fusion. In some embodiments, the internal control is a FFPE tissue section, blood, plasma, cells, nucleic acids, or oligonucleotides.

In some embodiments, when one or more MET fusions are detected in a biological sample from a cancer patient, the patient is expected to respond to a tyrosine kinase inhibitor, particularly a MET inhibitor such as crizotinib and cabozantinib.

### EXAMPLES

### Example 1

### Detection of MET Fusion by a Single-Amplified Target-Probe-BMB Hybridization Assay

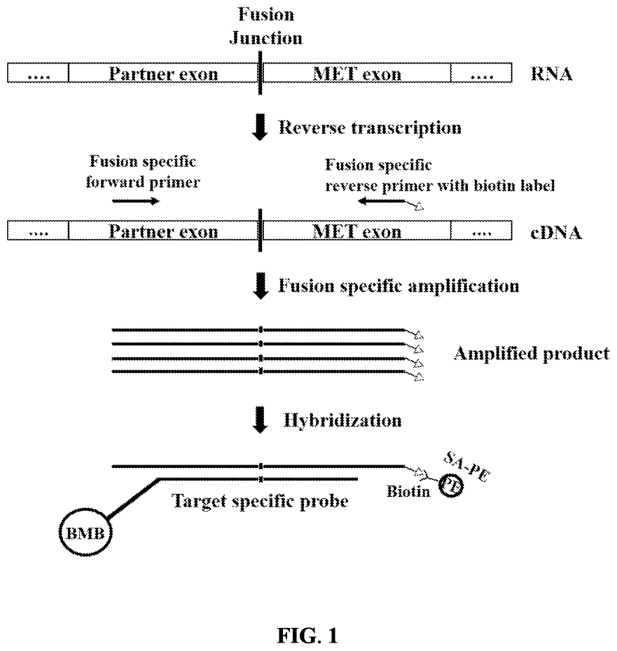

Single-amplified target-probe-barcoded magnetic bead (BMB) hybridization assay can simultaneously detect multiple possible MET fusion types in a single reaction. FIG. 1 shows the overall process of this assay, which includes the steps of obtaining RNA from a biological sample, reverse transcription of the RNA to obtain cDNA, PCR amplification of MET fusion regions of the cDNA (i.e., the target cDNA) using multiple MET fusion-specific primer pairs to obtain an amplified product of the target cDNA, probe hybridization with the amplified target cDNA using BMB-coupled probes and detection of the probe-bound product. Below is an example of this assay.

### Preparation of MET-Fusion Specific Probes

Prior to the assay, a probe targeting MET exon 14 skipping mutation was designed based on the nucleotide sequence of the fusion region in the RNA transcript of the MET gene (Table 2). The first 20 and the last 20 base pairs of the sequence in Table 2 are from the 5′ partner (exon 13 of the MET gene) and the 3′ partner (exon 15 of the MET gene), respectively. For hybridization at the site underlined in Table 2, one probe was designed to have the sequence listed in Table 3. The probe was synthesized and modified with an amine group at the 5′-end by IDT (Integrated DNA Technologies, Inc, Coralville, Iowa). Subsequently, the fusion specific probe was coupled with BMBs having a specific identification number via amine-carboxyl bonding, forming a “probe-BMB” complex.

TABLE 2

| MET | | | Sequence of the fusion region | SEQ ID |
|---|---|---|---|---|
| fusion ID | 5′ gene | 3′ gene | (from the 5′ end to the 3′ end) | NO |
| 001 | MET | MET | AAAGAGAAAGCAAATTAAAG | 75 |
| | exon 13 | exon 15 | ATCAGTTTCCTAATTCATCT | |

TABLE 3

| MET | Probe Sequence (from the | |
|---|---|---|
| fusion ID | 5′-end to the 3′-end) | BMB ID |
| 001 | GGAAACTGATCTTTAATTTG | 95 |
| | (SEQ ID NO: 38) | |

### PCR Amplification Using MET-Fusion Specific Primer Pair

To substitute clinical samples harboring MET fusion, oligonucleotides having the sequence of SEQ ID NO:75 (termed MET fusion oligo) were synthesized by IDT to be used as a positive control template. The MET fusion oligo was amplified by PCR with a MET fusion-specific primer pair shown in Table 4. This primer pair, capable of binding to the 5′-end and the 3′-end of the MET fusion oligo, was synthesized by IDT. The reverse primer in the primer pair was modified at the 5′-end with biotin for subsequent interaction with a streptavidin-phycoerythrin (SA-PE) conjugate (Thermo Fisher Scientific). The PCR was performed on Veriti™ 96-Well Thermal Cycler (Thermo Fisher Scientific) for 30 thermal cycles using Platinum Taq DNA polymerase High Fidelity (Thermo Fisher Scientific) according to the manufacturer's instructions.

TABLE 4

| MET | Sequence of the primer pair | | SEQ ID |
|---|---|---|---|
| fusion ID | from the 5′ end to the 3′ end) | | NO |
| 001 | Forward | AAGAGAAAGCAAATTAAAGATCAGTT | 76 |
| | Reverse | CTGTCAGAGGATACTGCAC | 77 |

### Probe Hybridization and Signal Detection

The amplified product of the MET fusion oligo was mixed with the probe-BMB in the same wells of a 96-well plate for hybridization. Hybridization was performed at 40° C. for 10-30 minutes with agitation at about 700 rpm. After the hybridization, a fluorescent SA-PE conjugate was added to the wells to bind the biotin of the amplified product, and the probe-BMBs were washed to remove unbound substances. An additional BMB (with an ID number of zero) bearing no probe was also added to the wells as a negative control. Finally, BioCode 2500 analyzer (Applied BioCode Inc., Taipei, Taiwan), equipped with a camera capable of both brightfield and fluorescence imaging, was used to read the barcodes of the BMBs and to detect the fluorescence signals of the BMBs.

Table 5 shows the fluorescence intensity of BMB95 and BMBO. According to Table 5, the fluorescence intensity of BMB95, indicating target-probe hybridization and the presence of MET exon 14 skipping, was significantly higher than that of the BMBO, indicating no MET fusion. The results show that the single-amplified target probe-BMB assay can be used to detect and distinguish MET fusion types.

TABLE 5

BMB ID

| Oligo ID | 0 | 95 |
|---|---|---|
| SEQ ID NO: 75 | 3 | 486 |

### Example 2

### Detection of MET Fusion by a Double-Amplified Target-Probe Hybridization Assay

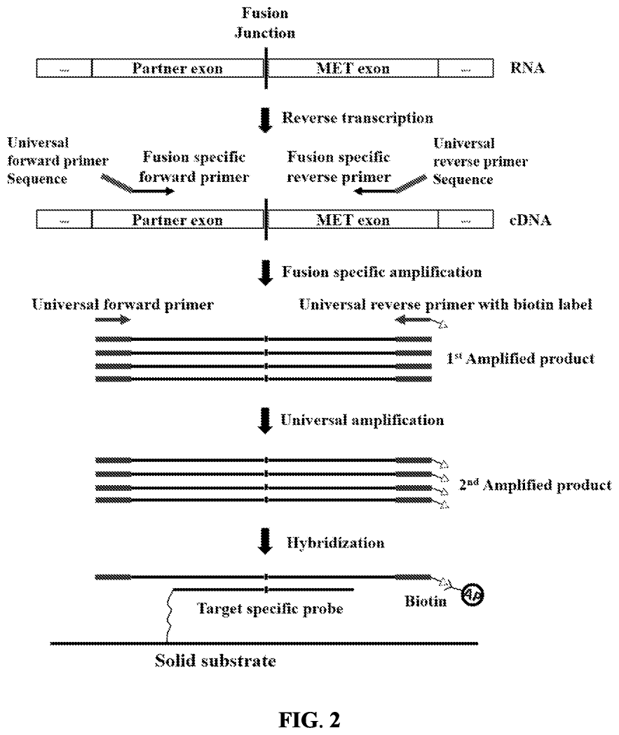

Double-amplified target-probe hybridization assay is another method designed for simultaneous detection of multiple possible MET fusions in a single reaction. FIG. 2 shows the overall process of this assay, which includes the steps of obtaining RNA from a biological sample, reverse transcription of the RNA to obtain cDNA, PCR amplification of MET fusion regions of the cDNA (i.e., the target cDNA) using multiple MET fusion-specific primer pairs to obtain a first amplified product of the target cDNA, PCR amplification of the first amplified product using a universal primer pair to obtain a second amplified product of the target cDNA, probe hybridization with the amplified target cDNA and detection of the probe-bound product. Below is an example of this assay.

### RNA Extraction and Reverse Transcription

Both DNA and RNA are extracted from a FFPE tissue specimen from a cancerous patient by using RecoverAll total nucleic acid isolation kit (Cat No: AM1975, Ambient Technologies) according to the manufacturer's instructions. Reverse transcription of 100 ng of total RNA is carried out at 42° C. for 30 to 60 minutes by using SuperScript cDNA synthesis kit (Cat No: 11754050, Invitrogen) and random hexanucleotide primers, and 10 pi, of cDNA product is obtained.

### PCR Amplification Using MET Fusion-Specific Primer Pairs

Each primer in the MET fusion-specific primer pair used in this assay is designed to have two segments. One segment, called a fusion specific segment, is used to bind the 5′-end or the 3′-end of the fusion sequence of one particular MET fusion. The other segment, called a universal segment, encompasses the nucleotide sequence of the universal primer to be used in the second round of PCR. The universal segment is always upstream, or at the 5′ position, relative to the fusion specific segment (FIG. 2). The universal primer may be any of the primers listed in Table 6, where each universal primer can be used as either the universal forward primer or the universal reverse primer.

TABLE 6

| Universal | Primer sequence (from the | SEQ ID |
|---|---|---|
| primer No. | 5′ end to the 3′ end) | NO |
| U01 | GTTTTCCCAGTCACGACGT | 78 |
| U02 | GCAAATGGCATTCTGACATCC | 79 |
| U03 | GCGGATAACAATTTCACACAGG | 80 |
| U04 | CGTCCATGCCGAGAGTG | 81 |
| U05 | CTTTATGTTTTTGGCGTCTTCCA | 82 |
| U06 | GACTGGTTCCAATTGACAAGC | 83 |
| U07 | GCGTGAATGTAAGCGTGAC | 84 |
| U08 | TGTAAAACGACGGCCAGT | 85 |
| U09 | AAGGGTCTTGCGAAGGATAG | 86 |
| U10 | GGGTTATGCTAGTTATTGCTCAG | 87 |

For fusion specific PCR, 7 μL of water is added to 10 μL of the cDNA product, and the resulting mixture (17 μL) is subsequently divided into 2-8 equal pools. The number of pools is decided based on primer performance. More specifically, the primer efficiency of each fusion specific primer pair is determined first, and the fusion specific primer pairs with similar efficiencies are mixed to form a single primer pool. Each primer pool, containing 1 to 40 fusion specific primers, is added into one pool of cDNA. The cDNA in each pool is then amplified on Veriti™ 96-Well Thermal Cycler (Thermo Fisher Scientific) for 15-30 thermal cycles using multiplex PCR kit (Cat No: 206143, Qiagen) according to the manufacturer's instructions, yielding a first amplified product in 10 μL.

### PCR Amplification Using a Universal Primer Pair

Since each fusion specific primer included the nucleotide sequence of a universal primer at the 5′ end, the first amplified products are able to be further amplified by PCR using a universal primer pair, including a universal forward primer with the sequence selected from SEQ ID NOs:78-87 and a universal reverse primer with the sequence selected from SEQ ID NOs:78-87. The universal reverse primer is biotinylated. For the second round of PCR, each pool of the first amplified products is diluted 100 folds in the final reaction mix and amplified on Veriti™ 96-Well Thermal Cycler (Thermo Fisher Scientific) for 25 thermal cycles by using Platinum SuperFi II PCR Master Mix (Cat No: 12368010, Invitrogen) according to the manufacturer's instructions, yielding a second amplified product in 10 μL.

### Probe Hybridization and Signal Detection

All pools of the second amplified products are combined to yield a mixture, which is placed in a 96-well PCR plate (Cat No: P46-4TI-1000/C, 4titude). The second amplified product is denatured at 96° C. for 5 minutes and transferred to pre-blocked wells, each of which is printed with an array of probe spots, including the spots of MET fusion-specific probes, the spots of control probes, and the spots of an anchor probe. Target-probe hybridization is performed at about 50° C. for 15 minutes with vibration. After the hybridization, the well is cooled and washed twice. A buffer containing a streptavidin-alkaline phosphatase conjugate is subsequently added to the wells to allow biotin-streptavidin interaction, and a substrate for the alkaline phosphatase is then added so that color products form at the position where a probe-target hybrid is present. By photographing the wells with a camera and identifying the position of color spots in the wells, the particular hybridization indicating the presence of a particular MET fusion can be determined. The position of color spots can be analyzed by a computer.

What is claimed is:

1. A kit for detecting MET proto-oncogene, receptor tyrosine kinase (MET) fusion, comprising:
at least two MET fusion-specific primer pairs; and
at least two probes each having a different nucleotide sequence selected from the group consisting of SEQ ID NOs:3, 4, 5, 23, 24 and any complementary sequence thereof.

2. The kit of claim 1, further comprising a universal primer pair, wherein a MET fusion-specific forward primer in each of the MET fusion-specific primer pairs further encompasses the nucleotide sequence of a universal forward primer in the universal primer pair, and a MET fusion-specific reverse primer in each of the MET fusion-specific primer pairs further encompasses the nucleotide sequence of a universal reverse primer in the universal primer pair.

3. The kit of claim 1, further comprising an additional probe having a nucleotide sequence selected from the group consisting of SEQ ID NOs:1-2, 6-22, 25-37 and any complementary sequence thereof.

4. The kit of claim 1, wherein a MET fusion-specific forward primer or a MET fusion-specific reverse primer in each of the MET fusion-specific primer pairs is connected to a detectable molecule.

5. The kit of claim 4, wherein the detectable molecule is a fluorescent molecule or an enzyme for a chromogenic reaction.

6. The kit of claim 2, wherein the universal forward primer or the universal reverse primer is connected to a detectable molecule.

7. The kit of claim 6, wherein the detectable molecule is a fluorescent molecule or an enzyme for a chromogenic reaction.

8. The kit of claim 1, wherein the at least two probes are immobilized on a microarray plate at different positions.

9. The kit of claim 8, wherein the microarray plate comprises a position indicator.

10. The kit of claim 1, further comprising a control probe for detecting a housekeeping gene.

11. The kit of claim 1, further comprising a reverse transcriptase and a DNA polymerase.

12. A method for detecting MET proto-oncogene, receptor tyrosine kinase (MET) fusion, comprising the steps of:
(a) obtaining ribonucleic acids (RNA) from a biological sample;
(b) performing reverse transcription of the RNA to obtain complementary deoxyribonucleic acids (cDNA);
(c) amplifying the cDNA with at least two MET fusion-specific primer pairs to obtain an amplified product;
(d) mixing the amplified product with at least two probes to obtain a probe-bound product, wherein each of the probes has a different nucleotide sequence selected from the group consisting of SEQ ID NOs:3, 4, 5, 23, 24 and any complementary sequence thereof; and
(e) detecting the probe-bound product to determine the presence of MET fusion.

13. The method of claim 12, wherein in step (c) the cDNA is amplified first with the at least two MET fusion-specific primer pairs and subsequently with a universal primer pair to obtain the amplified product, wherein a MET fusion-specific forward primer in each of the MET fusion-specific primer pairs further encompasses the nucleotide sequence of a universal forward primer in the universal primer pair, and a MET fusion-specific reverse primer in each of the MET fusion-specific primer pairs further encompasses the nucleotide sequence of a universal reverse primer in the universal primer pair.

14. The method of claim 12, wherein in step (d) the amplified product is mixed further with an additional probe having a nucleotide sequence selected from the group consisting of SEQ ID NOs:1-2, 6-22, 25-37 and any complementary sequence thereof.

15. The method of claim 12, wherein a MET fusion-specific forward primer or a MET fusion-specific reverse primer in each of the MET fusion-specific primer pairs is connected to a detectable molecule.

16. The method of claim 15, wherein the detectable molecule is a fluorescent molecule or an enzyme for a chromogenic reaction.

17. The method of claim 13, wherein the universal forward primer or the universal reverse primer is connected to a detectable molecule.

18. The method of claim 17, wherein the detectable molecule is a fluorescent molecule or an enzyme for a chromogenic reaction.

19. The method of claim 12, wherein in step (d) the at least two probes and the amplified product are mixed at a temperature between 35-50° C.

20. The method of claim 12, wherein in step (d) the at least two probes and the amplified product are mixed at a rotation speed between 700-1000 rpm.

21. The method of claim 12, wherein the at least two probes are immobilized on a microarray plate at different positions.
